(19) United States
(12) Patent Application Publication
(10) Pub. No.: US 2023/0285630 A1
(43) Pub. Date: Sep. 14, 2023

(54) PLASMA HYDROGEL THERAPY

(71) Applicant: UNIVERSITY OF SOUTH AUSTRALIA, ADELAIDE (AU)

(72) Inventors: Robert David SHORT, Shelfield (AU); Endre Jozsef SZILI, Atheistone (AU)

(73) Assignee: UNIVERSITY OF SOUTH AUSTRALIA, ADELAIDE (AU)

(21) Appl. No.: 18/052,988

(22) Filed: Nov. 7, 2022

Related U.S. Application Data
(63) Continuation-in-part of application No. 15/119,848, filed on Sep. 1, 2016, now abandoned.

(30) Foreign Application Priority Data
Feb. 18, 2014 (AU) 2014900507
Aug. 6, 2014 (AU) 2014903043

(51) Int. Cl. A61L 26/00

(52) U.S. Cl. CPC A61L 26/008; A61L 26/0052; A61L 26/0066; A61L 2300/10; A61L 2300/11

(57) ABSTRACT

Disclosed herein is a plasma treatment method comprising: providing a plasma source and a screen comprising a hydrogel and positioning the screen between the plasma source and a surface of a target to be treated with the plasma such that substantially all of the plasma from the plasma source passes through the screen prior to contacting the surface of the target and the screen reduces the concentration of one or more species from the plasma; and/or contacting a surface of a target to be treated with the gel composition comprising a gel forming material and a liquid phase comprising plasma activated liquid.

### PRIORITY DOCUMENTS

The present application is a continuation-in-part of U.S. Pat. Application Serial No. 15/119,848, filed Sep. 1, 2016, which is a National Stage of PCT No. PCT/AU2015/000087, filed on Feb. 18, 2015, which claims from the benefit of Australian Provisional Pat. Application No. 2014900507 titled “PLASMA SCREENS AND USES THEREOF,” filed on Feb. 18, 2014, and Australian Provisional Patent Application No. 2014903043 titled “PLASMA ACTIVATED HYDROGEL THERAPY,” filed on Aug. 6, 2014, the entire content of which are incorporated by reference herein.

### TECHNICAL FIELD

The present invention relates to the use of plasma in medical, therapeutic, veterinary, agricultural, environmental and related applications. In one form, the present invention relates to the use of materials to filter or screen plasma in medical and related applications. In another form, the present invention relates to plasma activated hydrogels, plasma hydrogels pre-loaded with a therapeutic agent and, in particular, the use of plasma activated hydrogels in therapeutic applications.

### BACKGROUND

Plasma is one of the four fundamental states of matter and can be produced in a number of ways such as by application of radio frequency, microwave frequencies, high voltage ac or dc to a gas. Plasma comprises photons, positive and negative ions, atoms, free radicals and excited and non-excited molecules. The range of species present in plasma has resulted in the use of plasma in a diverse range of applications including waste disposal, food processing, and plasma medicine.

Plasma has been used in medical applications for many years. For example, thermal plasmas have been used for sterilisation of equipment and implants, tissue destruction, cutting and cauterising.

More recently “non-thermal plasmas” (also referred to as “cold atmospheric plasmas” or “CAPs”) have enabled the extension of medical applications of plasma to the treatment of living tissue. Non-thermal plasmas are non-equilibrium plasmas in which the gas remains at relatively low temperature relative to the temperatures that are generated in thermal plasmas. Non-thermal plasmas are weakly ionised plasmas and comprise a highly active mix of oxygen, nitrogen and hydrogen radicals, ions, electrons, photons and ultraviolet (UV) radiation.

Amongst a range of other medical uses, non-thermal plasmas have been used in wound healing, blood coagulation and tissue generation. For example, Isbary et al. describe the use of non-thermal plasmas in the treatment of chronic wounds in patients (Isbary 2010, Isbary 2012). Non-thermal plasmas were shown to lead to a highly significant reduction in bacterial load in chronic wounds relative to standard wound care alone. These studies concluded that non-thermal plasmas are advantageous in wound care because the physical and chemical characteristics of plasmas allow them to penetrate small cavities, such as hair follicles, where other agents fail to reach. Furthermore, pathogen resistance is less likely to develop to non-thermal plasmas as plasma is thought to attack pathogens by a number of processes including reactive species, charging, permeabilisation, local energy deposition, and electroporation. Clearly, treatment with non-thermal plasmas is a promising development in wound care and other medical applications.

Whilst non-thermal plasmas contain potentially beneficial agents such as nitric oxide and hydrogen peroxide which can aid in the regeneration of tissue and stimulate wound healing they also contain harmful agents such as UV radiation, radicals and toxic gases (e.g. ozone). For example, hydroxyl radicals (readily produced by atmospheric-pressure plasmas) may be involved in all stages of carcinogenesis (Halliwell and Gutteridge, 2007; Nyskohus et al., 2013). Currently, it is not possible to easily remove the harmful agents from plasma (by changing the plasma treatment parameters) whilst enabling the delivery of the beneficial agents.

There is thus a need to provide systems and methods that reduce the amount of some species present in plasmas, whilst retaining the beneficial species, particularly for plasmas that are used in medical applications.

Furthermore, wounds are susceptible to infection by invading pathogens and any such infection tends to interrupt the normal wound healing process and can lead to the formation of chronic, non-healing wounds in which there is an abnormally prolonged healing phase, recurrence or non-healing of the wound (Wysocki, 1996). Wounds, and particularly chronic wounds, represent a major burden to healthcare systems around the world and significantly impact sufferers through a loss of mobility, long-term pain and decreased productivity.

Wound treatments typically involve physically covering the wound with a dressing so as to provide a physical barrier to the ingress of pathogens. A wide variety of materials are used to fabricate wound dressings and these range from simple gauze-type dressings to animal derived protein-type dressings such as collagen dressings. Advanced polymeric dressing materials that are able to maintain a moist wound environment have been shown to be more effective than gauze-type dressings in the treatment of chronic wounds. For example, synthetic dressings formed from polyurethane, polyvinylpyrolidone (PVP), polyethyleneoxide (PEO), polyvinyl alcohol (PVA) or polyacrylonitrile (PAN) can be modified to provide wound dressings with specific properties such as moisture retention and high fluid absorption. These properties promote healing by protecting wounds from infection and maintaining moisture levels in the wound. For example, Huang discloses in United States Patent No. 6,238,691 a three dimensional cross-linked polyurethane hydrogel wound dressing, which is absorptive, contours to a wound site and maintains the wound in a moist state to promote healing.

Therapeutic agents, such as those that impart antimicrobial or inhibitory activity, have also been used as additives in wound dressings. Silver based compounds (Arglaes and Acticoat dressings), chlorhexidine gluconate (Chlorhexidine Gauze Dressing BP), benzalkonium chloride (Band-Aid brand gauze dressing), parabens (NugelDressing), and PHMB (Kerlix and Excilon gauze dressings) have been incorporated into commercially available wound dressings in order to impart an antimicrobial or bioinhibitory property to the dressing.

There is also a need for wound dressings with occlusive, antibacterial and/or absorbent properties that can be applied to wounds to provide optimal conditions for healing.

### SUMMARY

The present inventors have investigated the use of plasma and hydrogels in medical and therapeutic applications, including the use of hydrogels loaded with a therapeutic agent whereby activation by plasma (direct or remote) results in delivery of the therapeutic agent.

Thus, provided herein is a plasma treated gel for use in medical and/or therapeutic applications. Also provided herein is a gel loaded with a therapeutic agent for use in conjunction with plasma in medical and/or therapeutic applications. Also provided herein is a use of a gel in medical and/or therapeutic applications of plasma.

According to a first aspect, there is provided a plasma treatment method comprising:
providing a plasma source and a screen comprising a hydrogel and positioning the screen between the plasma source and a surface of a target to be treated with the plasma such that substantially all of the plasma from the plasma source passes through the screen prior to contacting the surface of the target and the screen reduces the concentration of one or more species from the plasma; and/or
contacting a surface of a target to be treated with the gel composition comprising a gel forming material and a liquid phase comprising plasma activated liquid.

According to a second aspect, there is provided a plasma treatment method comprising:
providing a plasma source and a screen comprising a hydrogel and a therapeutic agent;
positioning the screen between the plasma source and a surface of a target to be treated with the plasma such that substantially all of the plasma from the plasma source passes through the screen prior to contacting the surface of the target and the screen reduces the concentration of one or more species from the plasma and activation of the screen by the plasma results in release of the therapeutic agent onto the surface of the target.

In the method of the second aspect, the therapeutic agent may (a) work in combination with the plasma treatment and/or (b) be released from the hydrogel upon plasma treatment and/or (c) enhance the plasma treatment.

The method of the second aspect may comprise multiple activations of the screen over time so as to release the therapeutic agent in stages.

In the method of the second aspect, the screen may be loaded with an agent that on plasma activation (direct or remote) enhances reactive oxygen species (ROS) production. For example, the screen may be loaded with a hydroxylamine compound that on plasma activation (direct or remote) enhances ROS production.

In the method of the second aspect, the screen may be loaded with pro-drugs that are unreactive until oxidized by hydrogen peroxide derived from plasma activation (Vadukoot, 2014).

The target to be treated may be an area of skin. Thus, according to a third aspect, there is provided a skin treatment method comprising:
providing a plasma source and a screen comprising a hydrogel and positioning the screen between the plasma source and a surface of the skin to be treated with the plasma such that substantially all of the plasma from the plasma source passes through the screen prior to contacting the surface of the wound and the screen reduces the concentration of one or more species from the plasma; and/or
contacting a surface of the skin to be treated with the gel composition comprising a gel forming material and a liquid phase comprising plasma activated liquid.

Furthermore, according to a fourth aspect, there is provided a skin treatment method comprising:
providing a plasma source and a screen comprising a hydrogel and a therapeutic agent;
positioning the screen between the plasma source and a surface of the skin to be treated with the plasma such that substantially all of the plasma from the plasma source passes through the screen prior to contacting the skin and the screen reduces the concentration of one or more species from the plasma and activation of the screen by the plasma results in release of the therapeutic agent onto the skin.

In one form, at least some of the present inventors have investigated the use of a screen comprising a transparent and flexible hydrogel that can be used to cover large areas and irregular shaped materials such as wound beds. The hydrogel, referred to as a plasma screen, allows the delivery of relatively long lived plasma species such as hydrogen peroxide through the material whilst it blocks the delivery of short lived plasma species such as hydroxyl radicals that may be harmful to the target site. The hydrogel can also be loaded with one or more therapeutic agent that may be released from the hydrogel when it is activated by plasma.

Thus, according to a fifth aspect, there is provided a screen for reducing the concentration of one or more species in plasma, said screen comprising a hydrogel.

According to a sixth aspect, there is a provided a plasma apparatus comprising a plasma source that generates a plasma jet, a screen comprising a hydrogel, said screen positioned relative to the plasma source so that the plasma jet passes through the screen prior to contacting a surface to be treated with the plasma jet and the screen reduces the concentration one or more species from the plasma, and a control system for controlling operation of the plasma source.

In certain embodiments of the sixth aspect, the screen further comprises a therapeutic agent.

According to a seventh aspect, there is provided a method for reducing the concentration of one or more species from plasma, the method comprising contacting a plasma screen comprising a hydrogel with a plasma such that the plasma passes through or partially through the hydrogel.

The screen and plasma apparatus described herein may be used in a range of applications including medical, therapeutic, veterinary, agricultural, environmental and related applications. In certain embodiments, the screen and plasma apparatus described herein are used in biological and medical applications of plasma including but not limited to dermatology (Heinlin, 2011), cancer treatment (Barekzi, 2013), and dentistry (Lee, 2009).

In another form, at least some of the present inventors have developed a wound dressing which is able to donate fluid to a wound surface whilst, at the same time, providing therapeutic properties using therapeutic agents generated by a plasma in the dressing and/or therapeutic agents present within the dressing that are released when the dressing is contacted with plasma.

According to an eighth aspect, there is provided a therapeutic gel composition comprising a gel forming material and a liquid phase comprising plasma activated liquid.

The therapeutic gel composition may be applied directly to wounds or it may be applied to a dressing or bandage which is then applied to wounds. The therapeutic gel composition can also be used in other therapeutic applications associated with skin disorders or ailments, such as burns, rashes, lesions, scars, acne, and the like.

According to a ninth aspect, there is provided a dressing for wounds, the dressing comprising a gel forming material and a liquid phase comprising plasma activated liquid.

According to a tenth aspect, there is provided a dressing for wounds, the dressing comprising a hydrogel activated by plasma.

Plasma activated liquid or hydrogel activated by plasma, refers to a liquid or hydrogel treated directly with a plasma discharge (i.e. the plasma glow directly contacting the liquid or hydrogel) or with the plasma effluent (i.e. without the plasma glow directly contacting the liquid or hydrogel). An example is plasma activated water (“PAW”) which is formed by treating water with a plasma discharge. As a result of the plasma treatment, there are changes in the water energy state and/or the physical, chemical and biological properties of the water. For example, there may be a decrease in the size of water clusters down to two to four molecules per cluster or even monomolecular, changes in light absorption spectra (visible IR and visible UV spectrum range), fluorescence spectra and NMR spectra, pH and ORP changes, generation of active components encapsulated in the PAW. PAW has been the subject of considerable therapeutic interest and has been shown to exhibit antimicrobial properties against a range of microbial species.

In the dressings of the present invention, the gel forming material and the liquid phase comprising plasma activated liquid interact with one another to form a hydrogel. The hydrogel may be formed from a natural polymer or a synthetic polymer.

The hydrogel can be formed by a number of methods. For example, the plasma activated liquid may be prepared (as described in detail later) and then mixed with the gel forming material to fabricate the hydrogel, which can then optionally be integrated into a wound dressing. Alternatively, a hydrogel can be formed first and integrated into a wound dressing. The hydrogel can then be treated with the plasma to form the dressing comprising the plasma activated liquid or other activated agents from the ingredients within the hydrogel. A secondary effect of using the latter method is that the plasma also sterilises the dressing.

According to an eleventh aspect, there is provided a method of treating a wound, the method comprising contacting the wound with a gel composition of the eighth aspect of the invention or a dressing of the ninth or tenth aspect of the invention.

According to a twelfth aspect, there is provided a use of a gel composition or dressing comprising a gel forming material and a liquid phase comprising plasma activated liquid for the treatment of a wound in a human or animal.

According to a thirteenth aspect, there is provided a gel composition or dressing comprising a gel forming material and a liquid phase comprising plasma activated liquid when used for the treatment of a wound in a human or animal.

According to a fourteenth aspect, there is provided a method of promoting the healing of a tissue wound in a human or animal by contacting the wound with a gel composition or dressing comprising a gel forming material and a liquid phase comprising plasma activated liquid.

According to a fifteenth aspect, there is provided a method of sterilising a wound in a human or animal and/or maintaining a wound in a human or animal in a sterile condition, the method comprising contacting the wound with a gel composition or dressing comprising a gel forming material and a liquid phase comprising plasma activated liquid.

### BRIEF DESCRIPTION OF DRAWINGS

Embodiments of the present invention will be discussed with reference to the accompanying drawings wherein:

FIG. 1 is a schematic of a plasma jet apparatus;

FIG. 2 shows photographs of the transparent and flexible plasma screen;

FIG. 3 is an illustration of the experimental set-up to monitor the plasma delivery of long lived plasma species (hydrogen peroxide in this particular example) through the plasma screen and into biological media;

FIG. 4 shows photographs and the corresponding absorbance values of the OPD solution after direct plasma jet treatment and neutral helium and plasma jet treatment through the plasma screen;

FIG. 5 shows plots for the helium plasma jet delivery of H2O2 species into PBS solution. Delivery of H2O2 was measured using the OPD-HRP reporter system. (a) is direct plasma treatment of the solution; this was compared to treatment through (b) a PVA screen and (c) through a gelatin screen;

FIG. 6 shows plots for the helium plasma jet delivery of nitrite/nitrate into PBS solution. Delivery of nitrite/nitrate was measured using the Griess Reagent reporter system. (a) Direct plasma treatment of the solution; this was compared to treatment through (b) a PVA screen and (c) through a gelatin screen;

FIG. 7 shows plots for the helium plasma jet delivery of ROS into GUVs. (a) is direct plasma treatment of the GUVs in PBS; this was compared to treatment through (b) a PVA screen and (c) through a gelatin screen;

FIG. 8 shows a plot of the relative amount of reactive oxygen and nitrogen species (RONS) delivered into phosphate buffered solution (PBS) of physiological pH 7.4 from gelatin made from non-plasma activated liquid (PBS) and gelatin made from plasma activated liquid (PBS);

FIG. 9 shows a plot of the relative amount of RONS delivered into PBS of physiological pH 7.4 from gelatin treated with helium and from gelatin activated by plasma; and

FIG. 10 shows a plot of the amount of RONS delivered into PBS of physiological pH 7.4 from Solosite™ treated with helium and from Solosite™ activated by plasma.

FIG. 11 shows a plot of the amount of RONS delivered into PBS of physiological pH 7.4 from Solosite™ treated with helium and from Solosite™ activated by plasma.

FIG. 12 shows the results of release of PVP-I by plasma activated hydrogels (right) vs control (left).

FIG. 13 shows the results of remote activated hydrogels placed on a lawn of bacteria on agar plates after 30 minutes (middle) and 1 hour (right).

FIG. 14 shows on the left-hand side four samples of skin that are all untreated and the sample-to-sample variation in resistance is between different skin samples. On the right-hand side there are four different treatments, (i) GAS only (no plasma), (ii) direct plasma (CAP), (iii) GAS (no plasma) plus hydrogel, and (iv) CAP plus hydrogel (Data obtained by Emily Owen and Toby Jenkins (Bath University)).

FIG. 15 shows the results of stratum corneum moisture measurements for skin samples that were treated with (i) GAS only (no plasma), (ii) direct plasma (CAP), (iii) GAS (no plasma) plus hydrogel, and (iv) CAP plus hydrogel (Data obtained by Emily Owen and Toby Jenkins (Bath University)).

FIG. 16 shows the results of trans epithelial water loss (TEWL) measurements for skin samples that were treated with (i) GAS only (no plasma), (ii) direct plasma (CAP), (iii) GAS (no plasma) plus hydrogel, and (iv) CAP plus hydrogel (Data obtained by Emily Owen and Toby Jenkins (Bath University)).

### DESCRIPTION OF EMBODIMENTS

Provided herein is a plasma treated gel for use in medical and/or therapeutic applications and a use of a gel in medical and/or therapeutic applications of plasma. Specifically provided herein is a plasma treatment method comprising: providing a plasma source and a screen comprising a hydrogel and positioning the screen between the plasma source and a surface of a target to be treated with the plasma such that substantially all of the plasma from the plasma source passes through the screen prior to contacting the surface of the target and the screen reduces the concentration of one or more species from the plasma; and/or contacting a surface of a target to be treated with the gel composition comprising a gel forming material and a liquid phase comprising plasma activated liquid. The method may be suitable for the treatment of skin. For example, the method may be suitable for the treatment of skin disorders including, but not limited to: wounds; lesions; tumors; inflammatory skin disorders such as dermatitis, contact dermatitis, atopic dermatitis, seborrheic dermatitis, nummular dermatitis, generalized exfoliative dermatitis, statis dermatitis, lichen simplex chronicus; disorders of hair follicles and sebaceous glands, such as acne, rosacea and rhinophyma, perioral dermatitis, and pseudo folliculitis barbae; and inflammatory reactions, such as drug eruptions, erythema multiforme, erythema nodosum, and granuloma annulare; rashes; blisters; abscesses; swelling; colorations; sores; and warts.

In one form, provided herein is a screen for reducing the concentration of one or more species from plasma, said screen comprising a hydrogel.

As used herein, the term “plasma” means plasma operated at around atmospheric pressure with the temperature of the plasma gas typically less than about 60° C. and ideally less than about 40° C. or less than about 37° C. upon contacting skin or tissue. Plasmas with higher gas temperatures may also be suitable. Higher gas temperatures are also suitable by adjusting the plasma exposure parameters: for example, a plasma gas temperature of 100° C. could be applied to a hydrogel by increasing the distance between the plasma source and the surface of the hydrogel or by decreasing the plasma exposure time.

The plasma can be formed using any plasma apparatus that generates a plasma stream that can be directed at a surface to be treated. The plasma apparatus may form a plasma jet, torch, needle or a dielectric barrier discharges (DBDs) such as a floating electrode configuration (Fridman, 2006) for treating a surface. Atmospheric pressure plasma jet devices are known in the art (see e.g. EP 0 921 713 A2, WO 98/35379 or WO 99/20809). Plasma jet devices can be fabricated in a multitude of electrode configurations and can be operated over a wide range of power and frequency (Hz to GHz) settings. A typical plasma jet device comprises two coaxially placed electrodes defining a plasma chamber there between. A plasma jet can be generated at an open end of the device by introducing a flow of gas at the other end of the device while a sufficient voltage is applied between the electrodes. A nozzle can be used at the open end to converge the plasma jet in order to obtain higher plasma densities. The plasma apparatus further comprises a power supply device for supplying electric power to the electrodes to produce plasma in the plasma chamber.

The plasma may be formed from an inert gas such as helium, argon or molecular gases such as oxygen, nitrogen, air or mixtures of any these gases. Optionally, the gas may also comprise an additive, such as an additive for improving the wound healing, improving the plasma characteristics or providing a sterilising effect.

The gas flow into the plasma chamber of the plasma apparatus is preferably controlled by a flow controller and/or an inlet valve which is arranged between a gas source and the gas inlet of the plasma apparatus.

Alternatively, the plasma can be operated in ambient air with no mechanical and/or physical control over the gas flow.

Optionally, the plasma apparatus has an ability to modulate an output to the electrodes. With this output modulation, it is possible to change the state of plasma. Note here that the output modulation refers to altering the output in characteristics to thereby change the plasma state-such as pulsating the output, increasing or decreasing the magnitude of output, turning on and off the output, changing output frequency or like processing.

In embodiments, the plasma has a gas temperature typically below 600C, when measured on the treated surface.

As discussed, we have found that a screen comprising a transparent and flexible hydrated gelatin film allows the delivery of long lived plasma species through the material whilst it blocks the delivery of harmful short lived plasma species (i.e. unwanted plasma species) such as hydroxyl radicals to the target site. The screen effectively prevents the passage of one or more plasma species or plasma effects from reaching a target site. Without intending to be bound by any specific theory we propose that hydrogels, such as gelatin, trap unwanted species such as UV radiation and short lived radicals within the gel structure and do not let them pass through. In this way, the composition of plasma that exits the hydrogel is different from the composition of the plasma that enters the hydrogel. Specific plasma species present in plasma and for which the concentration is preferably reduced include UV/VUV radiation, highly reactive oxygen species (ROS), and reactive nitrogen species (RNS). The plasma screen may also reduce or minimise one or more effects of the plasma on the target including, but not limited to, etching, ablation, dehydration, pressure, shear stress, temperature, pH, electrical currents, UV photons, positive and negative ions and atoms on the target site (Kong et al., 2009; Stoffels et al., 2008).

As used herein, the term “hydrogel” means a material which is not a readily flowable liquid and not a solid but a gel which is comprised of a gel forming material and water. The hydrogel may be formed by the use of a gel forming material which forms interconnected cells which binds to, entrap, absorb and/or otherwise hold water and thereby create a gel in combination with water.

The gel forming material that is used to form the hydrogel may be a natural or synthetic hydrophilic polymer material. Suitable natural materials include: gelatin; agarose; hypromellose; Matrigel; extracellular matrix proteins such as fibrin, fibronectin, collagen and collagen derivatives; polysaccharides, such as xanthan gum; sugars; celluloses and modified celluloses such as hydroxypropyl cellulose, sodium carboxymethyl cellulose and hydroxyethyl cellulose; and polycarboxylic acids.

Alternatively, the screen may comprise a non-porous and/or porous and cross-linked polymer and/or non-cross linked polymer material such as polyethylene oxide, polyvinyl alcohol, polyacrylic acid, polyvinyl pyrrolidone, polyacrylamidomethylpropanesulfonate, polycaprolactone (PCL), polyglycolic acid (and its derivatives) and copolymers thereof.

In some embodiments, the gel forming material comprises a commercial hydrogel selected from the group consisting of: AquaformTM, CurafilTM, GranugelTM, HypergelTM, Intrasite GelTM, Nu-GelTM, and Purolin gelTM (Jones and Vaughan, 2005).

In other embodiments, the gel forming material comprises a polymeric material selected from the group consisting of: poly(lactide-co-glycolide), poly(vinyl pyrrolidone), poly(vinyl alcohol), poly(hydroxyalkylmethacrylates), polyurethane-foam, and hydrocolloid and aliginate dressings (Boateng et al., 2008).

Commercially available amorphous hydrogels that can be used include: Anasept™ Antimicrobial Skin & Wound Gel (Anacapa Technologies, Inc.), 3M™ Tegaderm™ Hydrogel Wound Filler (3M Health Care), AmeriDerm Wound Gel (AmeriDerm Laboratories, Ltd.), AquaSite™ Amorphous Hydrogel Dressing (Derma Sciences, Inc.), Curasol™ Gel Wound Dressing (Smith & Nephew, Advanced Wound Biotherapeutics), Dermagran™ Amorphous Hydrogel Dressing (Derma Sciences, Inc.), DermaPlex™ Gel (MPM Medical, Inc.), DermaSyn™ (DermaRite Industries, LLC), DuoDERM™ Hydroactive Sterile Gel (ConvaTec), Excel™ Gel (MPM Medical, Inc.), Gentell Hydrogel (Gentell Wound and Skin Care), Hydrogel Amorphous Wound Dressing (McKesson Medical-Surgical), Hypergel™ Hypertonic Gel (Mölnlycke Health Care US, LLC), INTRASITE* Gel Hydrogel Wound Dressing (Smith & Nephew, Inc.), Kendall™ Amorphous Hydrogel (Covidien), LipoGel™ (Progressive Wound Care Technologies, Inc.), MacroPro™ Gel (Mölnlycke Health Care US, LLC), MPM Regenecare™ HA Spray (MPM Medical, Inc.), Normlgel™ Isotonic Saline Gel (Mölnlycke Health Care US, LLC), Purilon™ Gel (Coloplast Corp.), Regenecare™ HA (MPM Medical, Inc.), Restore™ Hydrogel (Amorphous) (Hollister Wound Care), SAF-GeI™ Hydrating Dermal Wound Dressing (ConvaTec), SilvaSorb™ Gel (Medline Industries, Inc.), SilverMed™ Amorphous Hydrogel (MPM Medical, Inc.), SilvrSTAT™ Antibacterial Wound Dressing Gel (ABL Medical, LLC), Skintegrity™ Hydrogel (Medline Industries, Inc.), SOLOSITE™ Wound Gel (Smith & Nephew, Inc.), Spand-Gel™ Primary Hydrogel (Medi-Tech International Corp.), and Woun′Dres™ Collagen Hydrogel (Coloplast Corp.).

In still other embodiments, the plasma screen may comprise a biological dressing (e.g. hyaluronic acid, chitosan and elastin) or a synthetic polymer (e.g. gauze or polysiloxanes) or a combination of both (e.g. IntegraTM bilayer matrix wound dressing).

In some embodiments, the hydrogel is in the form of a coating on a gauze pad, nonwoven sponge, rope and/or strip. In these embodiments, the screen comprises an impregnated hydrogel in which the hydrogel is coated onto a gauze pad, nonwoven sponge, rope and/or strip. The impregnated hydrogel may be formed by coating a gauze, sponge, rope or strip material with a suitable hydrogel, such as gelatin. Alternatively, a commercially available impregnated hydrogel of this type that can be used, such as: AquaSite™ Hydrogel Impregnated Gauze (Derma Sciences, Inc.), DermaGauze™ (DermaRite Industries, LLC), Gentell Hydrogel Impregnated Gauze (Gentell Wound and Skin Care), Hydrogel Impregnated Gauze Dressing (McKesson Medical-Surgical), Kendall™ Hydrogel Impregnated Gauze (Covidien), MPM GelPad™ Hydrogel Saturated Gauze Dressing (MPM Medical, Inc.), Restore™ Hydrogel Dressing (Impregnated Gauze) (Hollister Wound Care), Skintegrity™ Hydrogel Dressing (Medline Industries, Inc.), and SOLOSITE™ Conformable Wound Gel Dressing (Smith & Nephew, Inc.).

In some embodiments, the plasma screen comprises a sheet hydrogel in which a hydrogel is supported by a thin fibre mesh. The sheet hydrogel may be formed by coating a fibre mesh with a suitable hydrogel, such as gelatin, Alternatively, a commercially available sheet hydrogel can be used, such as: AquaClear® (Hartmann USA, Inc.), AquaDerm™ (DermaRite Industries, LLC), Aquaflo™ Hydrogel Dressing (Covidien), AquaSite™ Hydrogel Sheet (Derma Sciences, Inc.), Aquasorb™ and Border (DeRoyal), Avogel™ Hydrogel Sheeting for Scars (Avocet Polymer Technologies, Inc.), Comfort-Aid™ (Southwest Technologies, Inc.), CoolMagic™ Gel Sheet (MPM Medical, Inc.), Curasol™ Gel Saturated 4×4 Dressing (Smith & Nephew, Advanced Wound Biotherapeutics), Derma-Gel^{Tm} Hydrogel Sheet (Medline Industries, Inc.), Elasto-Gel™ (Southwest Technologies, Inc.), FLEXIGEL* Hydrogel Sheet Dressing (Smith & Nephew, Inc.), Hydrogel Sheet Dressing (McKesson Medical-Surgical), MediPlus™ Barrier Gel Comfort Border (MediPurpose, Inc.), MediPlus™ Barrier Gel Hydrogel Dressing (MediPurpose®, Inc.) NU-GEL™ Wound Dressing (Systagenix), Spand-Gel™ Hydrogel Dressing Sheets (Medi-Tech International Corp.), Toe-Aid™ (Southwest Technologies, Inc.), and XCell™ Cellulose Wound Dressing (Medline Industries, Inc.).

In specific embodiments, the hydrogel is gelatin. Gelatin can be obtained by the hydrolysis of collagen by boiling skin, ligaments, tendons, etc. A mixture of 2% gelatin in water forms a stiff hydrogel. The hydrogel may be formed by adding gelatin to water at an elevated temperature to dissolve the gelatin. The solution is then cooled and the solid gelatin components form submicroscopic crystalline particle groups which retain a considerable amount of water in the interstices.

The hydrogel will typically be transparent but it may also be opalescent.

The plasma screen comprising the hydrogel can take any shape or form. Indeed, the shape or form of the plasma screen may be selected to suit the intended use. In some embodiments, the plasma screen is a wound or skin dressing and in these embodiments the material is conveniently in the form of a sheet, layer or film. The sheet, layer or film may have any thickness range (but typically less than 1.5 mm). The thickness of the sheet, layer or film can be used to change the composition of the plasma that passes through the plasma screen. For example, a thicker sheet, layer of film is expected to remove more of the species in the plasma than a thinner sheet, layer or film.

The plasma screen can also take the form of a nozzle or plug that is configured to be inserted over the nozzle of the plasma jet assembly to filter the plasma generated species. In these embodiments, the plasma screen may comprise an ultra-thin polymer (i.e. <0.01 mm).

The plasma screen may be ultra-thin with an average thickness of from about 0.2 mm to about 0.3 mm, or from about 0.3 mm to about 0.4 mm, or from about 0.4 mm to about 0.5 mm. In certain embodiments, the plasma screen has an average thickness of less than about 0.2 mm and preferably less than about 0.1 mm. An ultra-thin screen may be applied by spraying, rolling, brushing, wiping or by other mechanical means, as an ultra-thin contiguous coating onto an open wound or intact skin or tissue to be treated. The plasma screen may be applied to form an occlusive seal with a surface to which it is applied, such as an open wound or intact skin or tissue.

The plasma screen may be part of a structure, such as a coating or layer on a porous polymer sheet (e.g. Mepore from Molnlycke), gauze pad, nonwoven sponge, a thin fibre mesh, rope or strip.

The hydrogel can be formed by mixing the gel forming material at a concentration of at least 1%, at least 2 %, at least 5%, at least 10 %, at least 20 %, at least 25 % or at least 30 % by weight with water or water with additives.

For wound treatment, a skin dressing comprising the hydrogel is applied over a wound or on a region of skin to be treated for cosmetic or therapeutic purposes. The plasma apparatus is configured so that the non-thermal plasma emitted therefrom contacts the surface of the hydrogel and the plasma that passes through the hydrogel contacts the wound or skin surface below to thereby sterilise the surface and improve the wound healing. We have shown that the plasma jet can deliver long lived plasma species such as hydrogen peroxide through the plasma screen after 5 min of treatment. Notably, the relative amount of hydrogen peroxide delivered after only 1 min of direct plasma jet treatment without the plasma screen was almost twice the amount delivered by the plasma jet via the plasma screen after 5 min of treatment. This indicates that the plasma jet delivers long lived plasma species (e.g. hydrogen peroxide) in a more controlled manner through the plasma screen in comparison to the direct plasma delivery without the plasma screen.

The plasma screen may comprise an additive such as a therapeutic agent. Useful therapeutic agents include antibiotics, antiseptic agents, antihistamines, hormones, steroids, therapeutic proteins, molecules, biologics, antibodies, anti-microbial peptides, oligonucleotides, RNAs, enzymes, growth factors, nucleic acids, wound healing agents, anti-inflammatory agents, anti-bacterial agents, antibiotics, anti-viral agents or other types of therapeutic agents to provide a desirable and/or beneficial effect. For example, antimicrobial agents such as silver based compounds, chlorhexidine gluconate, benzalkonium chloride, parabens, PHMB or PVPI-I can be loaded into the plasma screen and can be released in situ. For example, biologically active compounds such as growth factors and antimicrobial agents can be loaded into the plasma screen enabling the controlled delivery of therapeutic agents to a biological site in a spatially controlled manner. The therapeutic agent could also be in the form of a pro-drug that is unreactive until oxidised by hydrogen peroxide generated by the plasma. Suitable pro-drugs for this purpose are described in Vadukoot, 2014.

If desired, the therapeutic agent may be encapsulated within vesicles, microparticles, nanoparticles or dendrimers. Thus, other suitable additives include a vesicle, a vesicle encapsulating the agent, a micro- or nano- particle encapsulating the agent, a dendrimer encapsulating the agent, a molecule, a biologic, an antibody, an anti-microbial peptide, an oligonucleotide, an RNA, an enzyme, a growth factor, a nucleic acid, a wound healing agent, an anti-inflammatory agent, an anti-bacterial agent, an antibiotic, an anti-viral agent or other types of therapeutic agents to provide a desirable and/or beneficial effect. Without restriction, in the case of a therapeutic agent encapsulated within a vesicle, particle or dendrimer, the action of the plasma may be to rupture the vesicle, particle or dendrimer and release said agent. Alternatively, or in addition, the vesicle, particle or dendrimer may be biodegradable.

Advantageously, by varying the plasma treatment parameters (e.g. time), the plasma can be used to deliver specific doses of said agent. For example, this can be used to perform multi-treatments to deliver fractionated doses of the therapeutic agent. The plasma screen can also be used to used deliver the additive, such as a therapeutic agent as described above, through the screen over a wide area or a localised area.

Also provided herein is a plasma treatment method comprising providing a plasma source and a screen comprising a hydrogel and positioning the screen between the plasma source and a surface of a target to be treated with the plasma such that substantially all of the plasma from the plasma source passes through the screen prior to contacting the surface of the target and the screen reduces the concentration of one or more species from the plasma.

The plasma treatment method can be used for the treatment of wounds, living tissue or skin diseases or skin disorders or for sterilisation of a natural or artificial body orifice of a human or animal body.

Also provided herein is a plasma apparatus comprising a plasma source that generates a plasma jet, a screen comprising a hydrogel, said screen positioned relative to the plasma source so that the plasma jet passes through the screen prior to contacting a surface to be treated with the plasma jet and the screen reduces the concentration one or more species from the plasma, and a control system for controlling operation of the plasma source.

Also provided herein is a method for reducing the concentration of one or more species from plasma, the method comprising contacting a plasma screen comprising a hydrogel with a plasma such that the plasma passes through or partially through the hydrogel.

In another form, provided herein is a therapeutic gel composition comprising a gel forming material and a liquid phase comprising plasma activated liquid. Also provided herein is a dressing for wounds, the dressing comprising a gel forming material and a liquid phase comprising plasma activated liquid.

It will be evident that the gel compositions and dressings described herein are particularly useful for the treatment of wounds. However, the person skilled in the art will also readily appreciate that the gel compositions and dressings described herein could also be used in other therapeutic applications, particularly those associated with skin disorders or ailments, such as burns, rashes, lesions, acne, scars, wrinkles, and the like.

As used herein, the term “wound” refers to all types of tissue injuries, including those inflicted by surgery and trauma, including burns, as well as injuries from chronic or acute medical conditions, such as atherosclerosis or diabetes. The compositions and wound dressings described herein are useful for treatment of all types of wounds, including wounds to internal and external tissues.

As used herein, the term “hydrogel” means a material which is not a readily flowable liquid and not a solid but a gel which is comprised of a gel forming material and a liquid such as water. The hydrogel may be formed by the use of a gel forming material which forms interconnected compartments which bind to, entrap, absorb and/or otherwise hold water or other fluid and thereby create a gel in combination with water or the fluid. The hydrogel thus has a liquid phase with an interlaced polymeric component, with at least 10% to 90% of its weight as water.

Recently, plasma activated liquid including PAW has been the subject of considerable interest and PAW has been shown to exhibit antimicrobial properties against a range of microbial species (Traylor, et al., J. Phys. D: Appl. Phys. 44 (2011) 472001).

PAW is formed by treating water with a plasma discharge. As a result of the plasma treatment, there are changes in the water energy state and/or the physical, chemical and biological properties of the water. For example, there may be a decrease of in the size of water clusters down to two to four molecules per cluster or even monomolecular. So called “small cluster water” is reported to have numerous useful characteristics (e.g. U.S. Pat. No. 5,824,353 to Tsunoda et al.).

Treatment of aqueous liquids with plasma has also been shown to result in bactericidal activity of the liquid itself. For example, plasma treatment of sodium chloride (NaCl) solution and its immediate addition to Escherichia coli resulted in complete bacteria inactivation (≥ 7 log) after 15 min exposure time. With a 30 min delay between plasma treatment of liquid and its addition to the bacteria, a bactericidal effect was reduced but still detectable (Oehmigen, et al., Plasma Processes and Polymers 8 (10), 2011, 904-913).

Treatment of water with a plasma discharge also results in changes in light absorption spectra (visible IR and visible UV spectrum range), fluorescence spectra and NMR spectra, pH and ORP changes and generation of active components (e.g. nitrate species) encapsulated in the PAW structure. Plasma treatment also results in the generation of reactive oxygen and nitrogen species (RONS) and components, such as oxygen, hydrogen, hydroxyl, peroxide and nitrogen oxides in the form of ions and radicals.

A range of plasma devices can be used to activate the liquid or hydrogel dressing. These include, but are not limited to, plasma jets, plasma pencils, plasma needles, plasma torches, dielectric barrier discharges, floating dielectric barrier discharges, surface plasmas, microplasmas, plasma arrays and direct and indirect and hybrid plasmas. For example, dressings could be activated by a surface plasma dielectric barrier discharge just prior to use.

The plasma gas can be an inert gas, molecular gas, reactive gas or any mixtures of these.

The gel forming material used to form the hydrogel can be any macromolecular monomer or polymer that gels or otherwise thickens in situ to form a hydrogel. It may be a natural or synthetic hydrophilic material. Suitable natural materials include: gelatin; agarose; hypromellose; Matrigel; extracellular matrix proteins such as fibrin, fibronectin, collagen and collagen derivatives; polysaccharides, such as xanthan gum; sugars; celluloses and modified celluloses such as hydroxypropyl cellulose, sodium carboxymethyl cellulose and hydroxyethyl cellulose; and polycarboxylic acids.

Suitable synthetic materials include non-porous and/or porous and cross-linked polymers and/or non-cross linked polymer materials such as polyethylene oxide, polyvinyl alcohol, polyacrylic acid, polyvinyl pyrrolidone, polyacrylamidomethylpropanesulfonate, polycaprolactone (PCL), polyglycolic acid (and its derivatives) and copolymers thereof.

In some embodiments, the gel forming material comprises a commercial hydrogel selected from the group consisting of: AquaformTM, CurafilTM, GranugelTM, HypergelTM, Intrasite GelTM, Nu-GelTM, and Purolin gelTM (Jones and Vaughan, 2005).

In other embodiments, the gel forming material comprises a polymeric material selected from the group consisting of: poly(lactide-co-glycolide), poly(vinyl pyrrolidone), poly(vinyl alcohol), poly(hydroxyalkylmethacrylates), polyurethane-foam, and hydrocolloid and alginate dressings (Boateng et al., 2008).

Commercially available amorphous hydrogels that can be used include: Anasept™ Antimicrobial Skin & Wound Gel (Anacapa Technologies, Inc.), 3M™ Tegaderm™ Hydrogel Wound Filler (3M Health Care), AmeriDerm Wound Gel (AmeriDerm Laboratories, Ltd.), AquaSite™ Amorphous Hydrogel Dressing (Derma Sciences, Inc.), Curasol™ Gel Wound Dressing (Smith & Nephew, Advanced Wound Biotherapeutics), Dermagran™ Amorphous Hydrogel Dressing (Derma Sciences, Inc.), DermaPlex™ Gel (MPM Medical, Inc.), DermaSyn™ (DermaRite Industries, LLC), DuoDERM™ Hydroactive Sterile Gel (ConvaTec), Excel™ Gel (MPM Medical, Inc.), Gentell Hydrogel (Gentell Wound and Skin Care), Hydrogel Amorphous Wound Dressing (McKesson Medical-Surgical), Hypergel™ Hypertonic Gel (Mölnlycke Health Care US, LLC), INTRASITE* Gel Hydrogel Wound Dressing (Smith & Nephew, Inc.), Kendall™ Amorphous Hydrogel (Covidien), LipoGel™ (Progressive Wound Care Technologies, Inc.), MacroPro™ Gel (Mölnlycke Health Care US, LLC), MPM Regenecare™ HA Spray (MPM Medical, Inc.), Normlgel™ Isotonic Saline Gel (Mölnlycke Health Care US, LLC), Purilon™ Gel (Coloplast Corp.), Regenecare™ HA (MPM Medical, Inc.), Restore™ Hydrogel (Amorphous) (Hollister Wound Care), SAF-Gel™ Hydrating Dermal Wound Dressing (ConvaTec), SilvaSorb™ Gel (Medline Industries, Inc.), SilverMed™ Amorphous Hydrogel (MPM Medical, Inc.), SilvrSTAT™ Antibacterial Wound Dressing Gel (ABL Medical, LLC), Skintegrity™ Hydrogel (Medline Industries, Inc.), SOLOSITE™ Wound Gel (Smith & Nephew, Inc.), Spand-Gel™ Primary Hydrogel (Medi-Tech International Corp.), and Woun′Dres™ Collagen Hydrogel (Coloplast Corp.).

The gel composition may be used as is and applied directly to a wound. The hydrogel may be in the form of a hydrogel when it is applied to the wound. For example, the hydrogel may be applied to a wound in the form of a paste. Alternatively, the hydrogel can be formed in situ on the wound surface using a variety of methods. For example, a composition can be applied as a pre-gelled formulation of monomers, macromers, polymers, or combinations thereof, maintained as solutions, suspensions, or dispersions that form the hydrogel upon or shortly after application. A composition can be applied to a wound by a spray, such as via a pump or aerosol device and a stimulus can then be brought into contact with the pre-gelled composition, before, during, or after application of the composition to the wound, causing crosslinking or other thickening of the macromer or polymer to form the hydrogel.

Alternatively, the hydrogel may be in the form of a coating on a gauze pad, nonwoven sponge, rope and/or strip. In these embodiments, the dressing comprises an impregnated hydrogel in which the hydrogel is coated onto a gauze pad, nonwoven sponge, rope and/or strip. The impregnated hydrogel may be formed by coating a gauze, sponge, rope or strip material with a suitable hydrogel, such as gelatin. Alternatively, a commercially available impregnated hydrogel of this type that can be used, such as: AquaSite™ Hydrogel Impregnated Gauze (Derma Sciences, Inc.), DermaGauze™ (DermaRite Industries, LLC), Gentell Hydrogel Impregnated Gauze (Gentell Wound and Skin Care), Hydrogel Impregnated Gauze Dressing (McKesson Medical-Surgical), Kendall™ Hydrogel Impregnated Gauze (Covidien), MPM GelPad™ Hydrogel Saturated Gauze Dressing (MPM Medical, Inc.), Restore™ Hydrogel Dressing (Impregnated Gauze) (Hollister Wound Care), Skintegrity™ Hydrogel Dressing (Medline Industries, Inc.), and SOLOSITE™ Conformable Wound Gel Dressing (Smith & Nephew, Inc.).

In some embodiments, the dressing comprises a sheet hydrogel in which a hydrogel is supported by a thin fibre mesh. The sheet hydrogel may be formed by coating a fibre mesh with a suitable hydrogel, such as gelatin, Alternatively, a commercially available sheet hydrogel can be used, such as: AquaClear® (Hartmann USA, Inc.), AquaDerm™ (DermaRite Industries, LLC), Aquaflo™ Hydrogel Dressing (Covidien), AquaSite™ Hydrogel Sheet (Derma Sciences, Inc.), Aquasorb™ and Border (DeRoyal), Avogel™ Hydrogel Sheeting for Scars (Avocet Polymer Technologies, Inc.), Comfort-Aid™ (Southwest Technologies, Inc.), CoolMagic™ Gel Sheet (MPM Medical, Inc.), Curasol™ Gel Saturated 4×4 Dressing (Smith & Nephew, Advanced Wound Biotherapeutics), Derma-Gel™ Hydrogel Sheet (Medline Industries, Inc.), Elasto-Gel™ (Southwest Technologies, Inc.), FLEXIGEL* Hydrogel Sheet Dressing (Smith & Nephew, Inc.), Hydrogel Sheet Dressing (McKesson Medical-Surgical), MediPlus™ Barrier Gel Comfort Border (MediPurpose, Inc.), MediPlus™ Barrier Gel Hydrogel Dressing (MediPurpose®, Inc.) NU-GEL™ Wound Dressing (Systagenix), Spand-Gel™ Hydrogel Dressing Sheets (Medi-Tech International Corp.), Toe-Aid™ (Southwest Technologies, Inc.), and XCell™ Cellulose Wound Dressing (Medline Industries, Inc.).

In specific embodiments, the hydrogel is gelatin. Gelatin can be obtained by the hydrolysis of collagen by boiling skin, ligaments, tendons, etc. A mixture of 2% gelatin in water forms a stiff hydrogel. The hydrogel may be formed by adding gelatin to water at an elevated temperature to dissolve the gelatin. The solution is then cooled and the solid gelatin components form submicroscopic crystalline particle groups which retain a considerable amount of liquid in the interstices.

The composition or dressing can be prepared by adding a liquid phase comprising plasma activated liquid to the gel forming material. The term “a liquid phase comprising plasma activated liquid” is intended to encompass plasma activated water as well as plasma activated aqueous fluids and phases. The liquid phase may contain water and other additives such as buffers, pH adjusting agents, therapeutic agents and the like. For example, useful therapeutic agents include antibiotics, antiseptic agents, antihistamines, hormones, steroids, therapeutic proteins, molecules, biologics, antibodies, anti-microbial peptides, oligonucleotides, RNAs, enzymes, growth factors, nucleic acids, wound healing agents, anti-inflammatory agents, anti-bacterial agents, antibiotics, anti-viral agents or other types of therapeutic agents to provide a desirable and/or beneficial effect. If desired, the therapeutic agent may be encapsulated within vesicles, microparticles, nanoparticles or dendrimers. The plasma activated water can be prepared by treatment using a plasma jet, as previously described (Szili et al., J. Phys. D: Appl. Phys. 2014, 47, 152002). The plasma may be formed using helium, argon etc. The plasma treatment time will depend on a number of factors but using the previously described plasma jet a treatment of 1-30 minutes is suitable. Afterwards, the plasma activated water can then be mixed with the gel forming material in an amount of between about 1% (w/v) and 50% (w/v), such as about 1% (w/v), 2% (w/v), 3% (w/v), 4% (w/v), 5% (w/v), 6% (w/v), 7% (w/v), 8% (w/v), 9% (w/v), 10% (w/v), 11% (w/v), 12% (w/v), 13% (w/v), 14% (w/v), 15% (w/v), 16% (w/v), 17% (w/v), 18% (w/v), 19% (w/v), 20% (w/v), 21% (w/v), 22% (w/v), 23% (w/v), 24% (w/v), 25% (w/v), 26% (w/v), 27% (w/v), 28% (w/v), 29% (w/v), 30% (w/v), 31% (w/v), 32% (w/v), 33% (w/v), 34% (w/v), 35% (w/v), 36% (w/v), 37% (w/v), 38% (w/v), 39% (w/v), 40% (w/v), 41% (w/v), 42% (w/v), 43% (w/v), 44% (w/v), 45% (w/v), 46% (w/v), 47% (w/v), 48% (w/v), 49% (w/v) or 50% (w/v). We have found about 10% (w/v) gelatin is suitable. The gel forming material is then allowed to interact with the liquid phase to form a hydrogel.

Alternatively, the gel forming material can be treated with water or aqueous fluid to form a hydrogel which is subsequently plasma treated using a plasma jet as described above for a time of about 1 minute to 10 minutes. In the case of a gelatin hydrogel, a plasma treatment time of about 5 minutes was suitable.

The dressing comprising the hydrogel can take any shape or form. Indeed, the shape or form of the dressing may be selected to suit the intended use. For wound or skin dressing the dressing is conveniently in the form of a sheet, layer or film. The sheet, layer or film may have any thickness range.

The substrate of the wound dressing may be a commercially available wound dressing or any flexible, non-toxic fabric that has sufficient structural integrity to withstand normal handling, processing and use. Suitable materials for the substrate include, but are not limited to, a woven or non-woven cotton, nylon, rayon, polyester or polyester cellulose fabric. A non-woven fabric may be spun-bonded, spun-laced, wet-laid or air-laid.

The compositions and dressings described herein provide for effective wound healing, moisture management capability, antimicrobial activity, and biocompatibility. For example, the compositions and dressings described herein provide high moisture donation and absorption capabilities which are particularly desirable for optimal wound healing. The incorporation of plasma activated liquid into the composition and dressing further enhances the healing process by combating or preventing microbial infections.

It will be evident from the foregoing description that the gel composition or dressing can be used for the treatment of a wound in a human or animal.

Also provided herein is:
a gel composition or dressing comprising a gel forming material and a liquid phase comprising plasma activated liquid when used for the treatment of a wound in a human or animal;
a method of promoting the healing of a tissue wound in a human or animal by contacting the wound with a gel composition or dressing comprising a gel forming material and a liquid phase comprising plasma activated liquid; and
a method of sterilising a wound in a human or animal and/or maintaining a wound in a human or animal in a sterile condition, the method comprising contacting the wound with a gel composition or dressing comprising a gel forming material and a liquid phase comprising plasma activated liquid.

Whilst the present invention is primarily concerned with the treatment of human subjects, the gel composition or dressing could also be used on non-human subjects, particularly mammalian subjects such as dogs, cats, livestock and horses for veterinary purposes.

Advantageously, the gel compositions and dressings described herein can be used in treatment of burns and scalds. The sterility of a composition or dressing used in these applications is important and an advantage of the compositions, dressings and methods described herein is that the use of plasma is a very good way of sterilising materials for dressing and delivery of RONS is expected to help keep the wound environment sterile.

Dressings as described herein may be available as pre-packaged, plasma activated dressings that aids the rate of healing. For example, dressings comprising a plasma activated hydrogel can be packaged under an inert atmosphere. It is possible that the dressings could be re-activated or further activated upon exposure to direct sunlight for example.

### EXAMPLES

### Example 1 - Plasma Jet Assembly for Plasma Screen

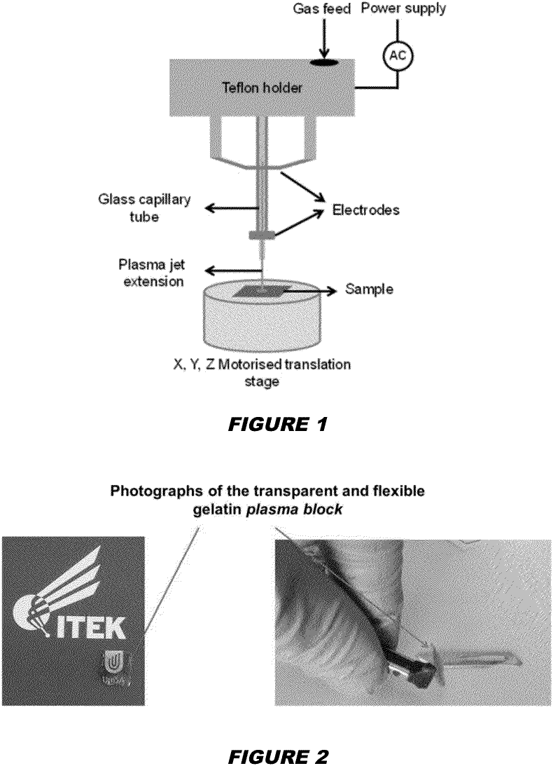

The plasma jet assembly consisted of a glass capillary tube with an inner diameter of 1 mm that was surrounded by two external hollow electrodes separated 4 mm apart (FIG. 1). The plasma was operated with 100 ml min-1 of helium at an applied voltage potential of 5.5 kVpeak-peak and a frequency of 10 kHz. These operational conditions produced a plasma plume of 10 mm in length. Plasma treatment was carried out at 2 and 3 mm separation distances between the end of the glass capillary tube and the surface of the hydrogel screen.

### Example 2 - Testing Plasma Screen

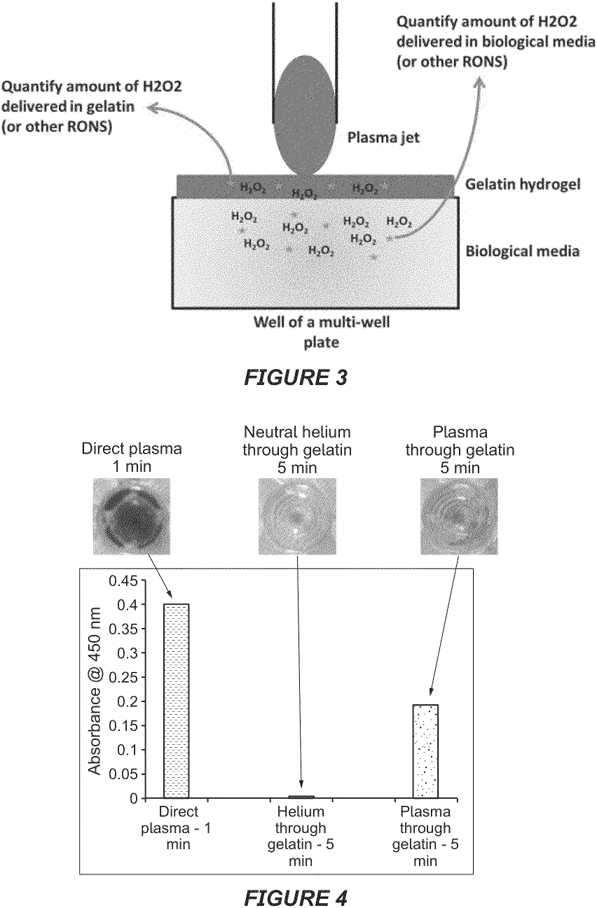

To test proof of principle we used a commonly employed a horseradish peroxidase (HRP) - hydrogen peroxide - o-Phenylenediamine (OPD) reporter system. HRP catalyses the oxidation of OPD in the presence of hydrogen peroxide converting the colourless OPD product into a yellow coloured product. The intensity of the yellow coloured product is directly proportional to the amount of hydrogen peroxide in the system which can be monitored spectrophotometrically by recording the absorbance of the solution at a wavelength of 450 nm. A thin sheet (approximately 1-2 mm thickness) of the plasma screen was placed over the top of the wells of a 96-well microplate containing 400 µl of an OPD/HRP pH 7.4 buffered solution (FIG. 3).

FIG. 4 shows that the plasma jet delivered hydrogen peroxide into the buffered solution through the plasma screen after 5 min of treatment. Hydrogen peroxide was not delivered into the solution by the control treatment of 5 min neutral helium gas flow. The relative amount of hydrogen peroxide delivered into the solution after only 1 min of direct plasma jet treatment without the plasma screen was almost twice the amount delivered by the plasma jet into the solution via the plasma screen after 5 min of treatment. This indicates that the plasma jet delivers hydrogen peroxide into the solution in a more controlled manner through the plasma screen in comparison to the direct plasma delivery of hydrogen peroxide without the plasma screen.

### Example 3 - Preparation of PVA and Gelatin Plasma Screens

The following plasma screens were prepared and investigated.

A 10% PVA hydrogel was prepared by dissolving 0.1 mg/ml polyvinyl alcohol (PVA) (Cat# 363065, Sigma-Aldrich) in phosphate buffered saline (PBS) solution (Cat# P4417, Sigma-Aldrich). A hot water bath at 200° C. with continuous stirring for 45-50 minutes was used to uniformly dissolve PVA in buffer. The hydrogel solution was allowed to settle at 90° C. for half an hour.

Thin PVA Screens were prepared by pouring the hydrogel solution in a petri dish covered with para-film. The petri dish was kept at -9° C. overnight. After the film was set, it was stored at 4° C. prior to use. A PVA screen of 1-1.3 mm thickness was used for this study.

A 5% gelatin hydrogel was prepared by dissolving 0.05 mg/ml Gelatin (Cat# G1890, Sigma-Aldrich) in PBS. A hot water bath at 200° C. with continuous stirring for 15-20 minutes was used to uniformly dissolve Gelatin in buffer. The solution was allowed to settle at 90° C. for half an hour.

Thin gelatin screens were prepared by pouring the hydrogel solution in a petri dish covered with para-film. The petri dish was kept a 4° C. overnight. A gelatin screen of 1-1.3 mm thickness was used for this study.

Example 4 - Use of the PVA and gelatin plasma screens for controlled delivery of hydrogen peroxide (H₂O₂)

A biological indicator comprising of 18.5 mM ortho-phenylenediamine (OPD) (Cat# P9029, Sigma-Aldrich) and 4 µg/ml horseradish peroxidase (HRP) (Cat# P6782, Sigma-Aldrich) prepared in PBS was utilised to monitor the plasma delivery of hydrogen peroxide (H₂O₂) through the Plasma Screen and into the buffer solution. This involved dispensing 400 µl of the indicator into a well of a 96-well multi-well format. The screen was placed on top of the solution and a plasma jet was directed down towards the screen so that the visible glow contacted the screen.

A helium jet was used in this study, which was reported in our previous studies (e.g. Hong et al, J.Phys.D:App.Phys. 47 (2014) 362001). Briefly, the operational parameters were: Voltage = 5.5 kVₚₑₐₖ₋ₚₑₐₖ; Frequency = 10 kHz; and treatment distance between the end of the glass tube of the plasma jet assembly and surface of the Screen was less than 1 mm, so that the plasma plume extension touches the Screen surface. Delivery of H₂O₂ through the screen into PBS was compared to direct delivery into the PBS. For direct delivery into PBS the treatment distance between the end of the glass tube of the plasma jet assembly and surface of the Screen was 1 mm.

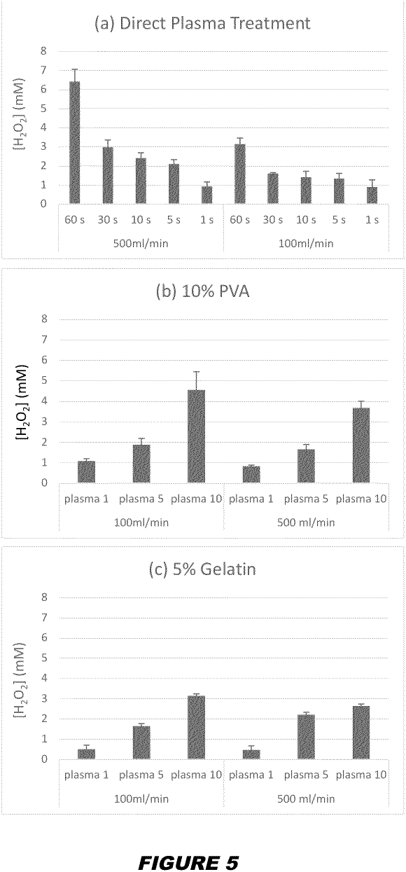

The results are shown in FIG. 5. The figure shows that the rate of H₂O₂ generation in the buffered solution is much higher for (a) the direct plasma treatment (without the screen) compared to (b and c) the plasma treatment through the screen. The data show that the rate of H₂O₂ delivered to the target material or solution (in this case PBS) is determined by the composition of the plasma screen, treatment time and the He gas flow rate.

### Example 5 - Use of the Plasma Screen for Controlled Delivery of Nitrite/Nitrate

We used 50 mg/ml Griess reagent (Cat# G4410, Sigma-Aldrich) prepared in PBS to monitor the plasma delivery of nitrite and nitrate through the plasma screen and into PBS. The treatment parameters were kept exact same as for H2O2 (Example 4).

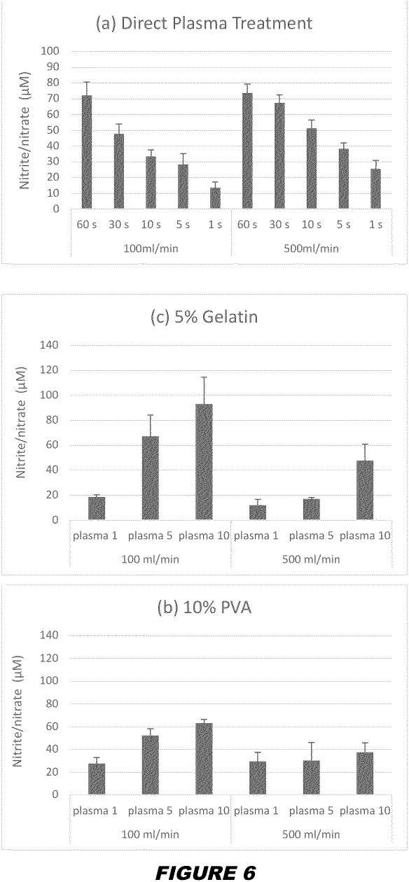

The results are shown in FIG. 6. The figure shows that the rate of nitrite/nitrate generation in PBS is much higher for (a) the direct plasma treatment (without the Screen) compared to (b and c) the plasma treatment through the Screens. The data show that the rate of nitrite/nitrate delivered to the target material or solution (in this case PBS) is determined by the composition of the Plasma Screen, treatment time and the He gas flow rate.

### Example 6 - Use of the Plasma Screen for Controlled Delivery of Reactive Oxygen Species (ROS) Into Cells

We used Giant Unilamellar Vesicles (GUVs) as a synthetic cell model. Phospholipid membrane GUVs encapsulating a chemical ROS reporter (2,7-dichlorodihydrofluorescein, DCFH) was utilised to study the plasma delivery of ROS into vesicles (and by inference, cells). GUVs were synthesised using a procedure reported elsewhere (Hong et al., J. Phys. D: Appl. Phys. 47 (2014) 36200).

The plasma treatment parameters and conditions are the same as described above.

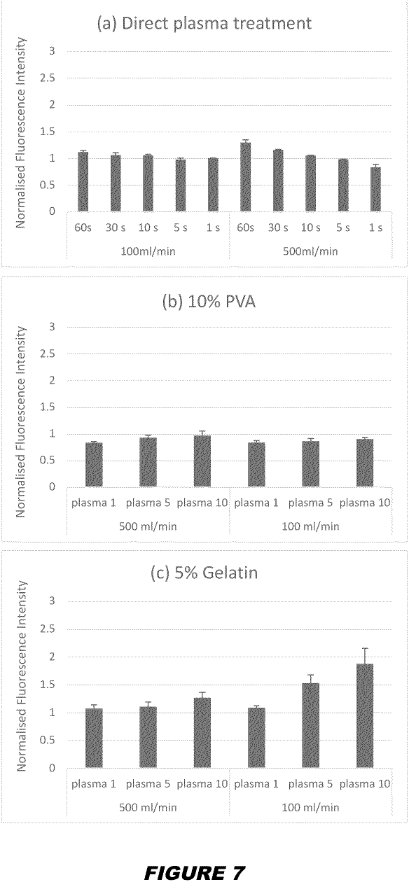

The results are shown in FIG. 7. The figure shows that the rate of ROS delivery into the GUVs (and by inference, cells) is much higher for (a) the direct plasma treatment (without the Screen) compared to (b and c) the plasma treatment through the Screen. The data show that the rate and quantity of ROS delivered to the tissue model (in this case GUVs) is determined by the composition of the Plasma Screen, treatment time and the He gas flow rate.

### Example 7 - Preparation of a Gelatin Hydrogel Using Plasma Activated Liquid

In this method the plasma is applied to the treatment of a liquid (such as water or buffered solutions). This (plasma-activated) liquid is subsequently used to fabricate a hydrogel, which can then be integrated into a wound dressing.

To manufacture the plasma-activated gelatin hydrogel, 5 ml of PBS was treated in the well of a 6-well multi-well plate using the plasma jet. The plasma jet source has already been described (E. J. Szili, J. W. Bradley, R. D. Short, J. Phys. D: Appl. Phys. 2014, 47, 152002). The treatment conditions were as follows: treatment distance (separation between the end of the glass capillary tube of the plasma jet assembly and the top of a 6-well multi-well plate) = 5 mm; gas type and flow rate = helium at 850 ml/min; applied voltage = 5.5 kVₚₑₐₖ₋ₚₑₐₖ; treatment time = 30 min. Afterwards, the treated solution was mixed with 10% (w/v) gelatin and the gelatin was allowed to dissolve at 40° C. for 1 h. The dissolved gelatin solution was dispensed in 100 µl aliquots into wells of a 96-well multi-well plate. The plate was placed into a sealed plastic bag to prevent dehydration and refrigerated at 4° C. for 12 h to set the gelatin.

### Example 8 - Plasma Treatment of a Gelatin Hydrogel to Form a Hydrogel Comprising Plasma Activated Liquid

In this method a hydrogel is first fabricated and integrated into a wound dressing. The hydrogel is then treated with the plasma to form the plasma-activated bandage. A secondary effect of using this method is that the plasma also sterilises the bandage.

To manufacture the plasma-activated gelatin hydrogel, 100 µl of gelatin was set into wells of a 96-well multi-well plate as described above except untreated PBS was used to make the gelatin (instead of the plasma activated PBS). The gelatin was subsequently treated with the plasma jet as described above using the following treatment conditions: treatment distance = 5 mm; gas type and flow rate = helium at 850 ml /min; applied voltage = 5.5 kVₚₑₐₖ₋ₚₑₐₖ; treatment time = 5 min.

### Example 9 - Assessment of Plasma Activation

To assess if a hydrogel (suitable for a dressing) could be activated by plasma, the relative amount of RONS loaded into a gelatin gel was analysed. A reporter dye 2,7-dichlorodihydrofluorescein (DCFH) was used for this study. This was obtained in a diacetate form. The dye was deacetylated in 10 mM NaOH for 30 min at 25° C. Afterwards, 10 ml of PBS at pH 7.4, is added to neutralise the solution.

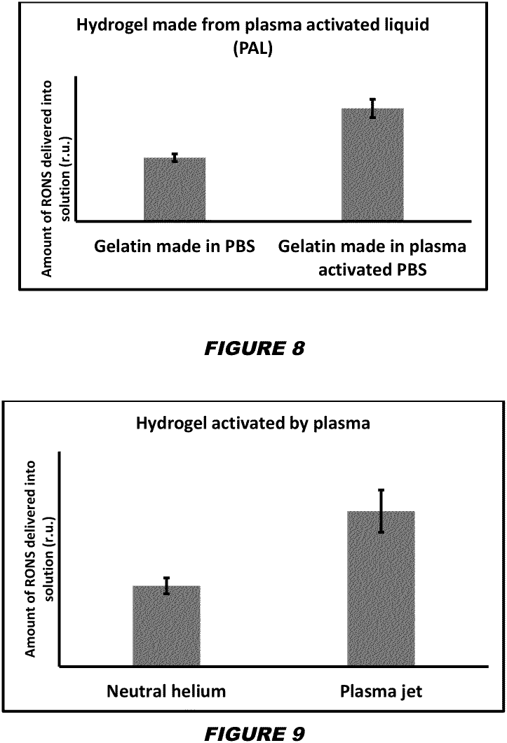

The release of the RONS into PBS was monitored by adding 200 µl of the prepared DCFH solution to the test wells containing the plasma activated gelatin. The DCFH solution was incubated in the wells for 10 min at 25° C. in the dark. A 100 µl aliquot of the DCFH solution was then transferred into a fresh well for measurement. Upon oxidation by RONS, non-fluorescent DCFH is converted to the highly fluorescent 2,7-dichlorofluorescein (DCF) product. Fluorescence of the test solution was measured using a BMG Labtech Fluostar Omega microplate reader. Fluorescence measurements were recorded at λ_{excitation} of 485 nm and λₑₘᵢₛₛᵢₒₙ of 520 nm. The fluorescence intensity is relatively proportional to the amount of RONS released by the plasma activated gelatin into the test solution. FIG. 8 shows that a hydrogel formed from plasma activated liquid can be used to deliver RONS into PBS and FIG. 9 shows that a hydrogel activated directly by plasma can be used to deliver RONS into PBS.

### Example 10 - Plasma Treatment of Hydrogel to Form a Hydrogel Comprising Plasma Activated Liquid

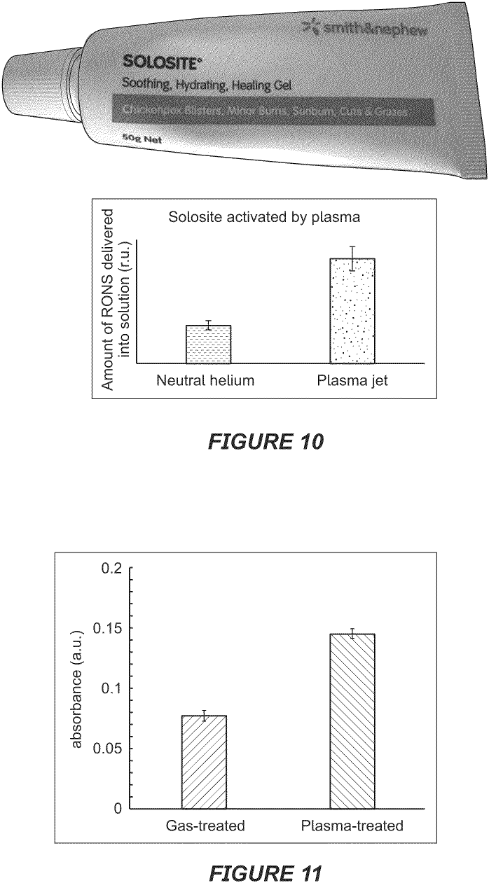

In another demonstration, a 100 µl volume of a commercially available wound healing gel (Solosite™, Smith & Nephew, hydrogel ingredient carmellose sodium (i.e. sodium carboxymethyl cellulose)) was treated with the plasma jet in wells of a 96-well multi-well plate using the same treatment parameters described above. Similar to the gelatin gel, Solosite™ gel was readily activated by the plasma jet and could be used to deliver RONS into PBS at physiological pH (FIG. 10).

### Example 11: Plasma Treatment Using a Hydrogel Containing PVP-I

The release of PVP-I from a 1 mm thick agarose hydrogel film when exposed to plasma multijet device was investigated. The agarose hydrogel film was placed on the top of a microwell containing 375 µl of deionised water in a 24-well plate. The PVP-I loaded hydrogel was treated with a multijet argon plasma at a distance of 1 mm for 3 minutes. A negative control under the same parameters but with argon gas only (no plasma) was also performed. The delivery of PVP-I from the hydrogel into the well was confirmed by formation of yellow colour in the water. After the treatment, the hydrogel was removed and a100 µl aliquot from the multiwell was transferred to a new 96-well plate for absorbance measurement at 400 nm. As show in FIG. 11, a two-fold increase in the absorbance values was observed upon treatment with plasma when compared to gas-only.

### Example 12: Plasma Treatment Using a Hydrogel Containing Pro-Drugs That Are Unreactive Until Oxidized by Hydrogen Peroxide

Acetyl donor prodrugs, such as those described in “Cold Plasma Generation of Peracetic Acid for Antimicrobial Applications” Volume 11, Issue 4, 2021, pp. 73-84 or Szili EJ et al., 2021 can be loaded into hydrogels and the hydrogels activated by plasma as described herein.

### Example 13: Plasma Treatment Using a Hydrogel Containing Compounds That Enhance ROS Production

Hydrogels can be loaded with any one or more of the following, all of which are expected to enhance ROS production:
Organoboron compounds: H₂O₂ activated organoboron compounds for medical applications (Saxon, 2022)
Hydroxylamine compounds (HA)
   o HA + H₂O₂can produce on-demand OH radicals
   o HA + H₂O₂in presence of a catalyst such as HRP can produce HNO (nitrous compound known for bacterial killing)
   o Enhanced catalytic H₂O₂Production from HA oxidation
Polyacrylonitrile (PAN): Enhanced antimicrobial activity of H₂O₂ using PAN catalyst (Boateng 2011)
Ferric ion compounds to enhance the production of H₂O₂ and reduce plasma treatment time
NaOCl: With H₂O₂ from plasma to create HOCl (Djimeli et al., 2014)
KI/KSCN
   o H₂O₂-activated peroxidase-catalyzed systems: H₂O₂ and its combinations with potassium iodide, with potassium thiocyanate and with both (H₂O₂/KI/KSCN) (Tonoyan 2017)
Periodate compounds
   o Production of ROS by the reaction of periodate and hydroxylamine for rapid removal of organic pollutants and waterborne bacteria (Sun 2020)
   o Periodate- H₂O₂ Mixture as strong oxidant (Kim 2022)

### Example 14 - Remote Plasma Activation of Hydrogels

4.5% PVA hydrogels with and without ROS enhancer (tetraacetylethylenediamine (TAED)/ pentaacetate glucose (PAG)) were double crosslinked in a 60 mm or 90 mm petri dish. The hydrogels in the petri dish were submerged in 5~10 mL aqueous solution (water, PBS or RONS enhancing solutions) and remotely activated using an Ar and He plasma jet at 9KV and 23.5 KHz with gas flow rate 0.8. The activation time was varied between 10 minutes and 60 minutes. Pre-activated hydrogels were stored at 4° C. up to 96 hours.

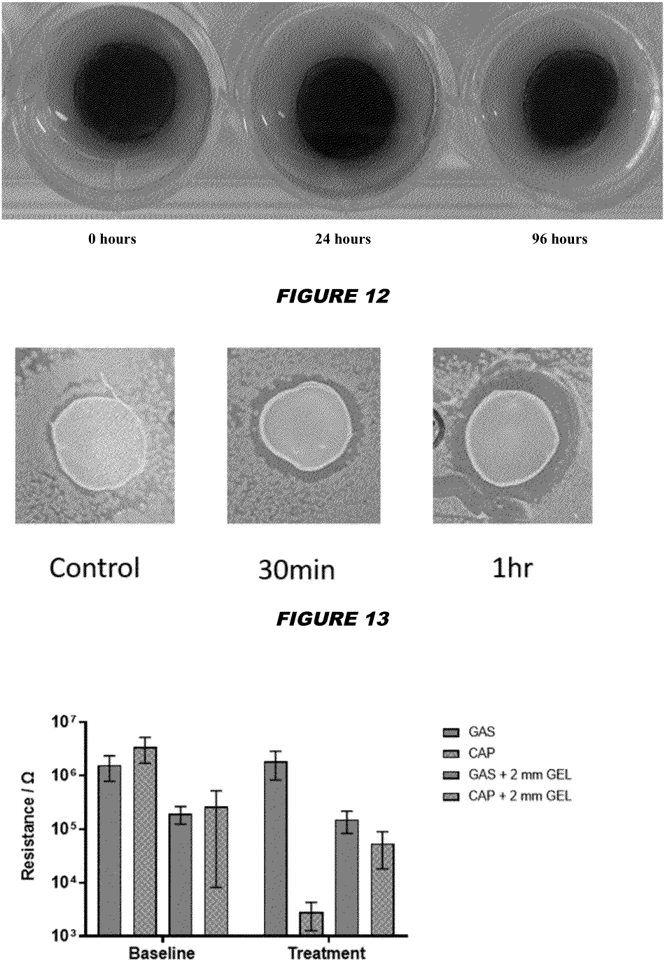

RONS retained in the remote plasma activated hydrogels were tested using KI-starch activation whereby colour indicates KI-Starch oxidation by RONS retained in remote activated hydrogels. The results (FIG. 12) show that remote plasma activated hydrogels activate starch-KI to a comparable degree up to 96 hours post treatment.

To assess antimicrobial efficacy of the remote plasma activated hydrogels the zone of inhibition was investigated. 100 µl of overnight culture of Escherichia coli (e. coli) was plated on nutrient agar plates to produce an end concentration of 1×10⁶ CFU/mL and spread out evenly. Remote activated hydrogel dressings were placed onto the bacterial lawn, treated side down and incubated at 37° C. for 24 hr. The results (FIG. 13) show that the normalised zone of inhibition (normalised to the size of the hydrogel), 30 min treated dressing produce 0.3 a.u. zone of inhibition and the 1 hr treated dressings produce 0.53 a.u. zone of inhibition.

### Example 15 - Assessing Skin Barrier Function After CAP Treatment Via EIS

Impedance spectroscopy was used to show that the resistance of skin changes on direct exposure to plasma after exposure of ex vivo porcine skin to Cold Atmospheric Plasma (CAP). The use of a PVA gel as a barrier between the skin and CAP was also assessed to see if this reduces the plasma-induced skin damage.

Impedance parameters: A PalmSens 4 potentiostat was used to measure impedance spectroscopy at a frequency range of 50000 - 0.1 Hz. An input AC voltage of 0.1 V was inputted. Skin impedance was measured using a two-electrode method; one ECG electrode connected to the working electrode and the other ECG electrode connected to the counter electrode, combined with the reference electrode. The chosen ECG electrodes were Red Dot 3 M pre-gelled Ag/AgCl electrodes.

The impedance data was fitted to an R 1 (R 2 Q) equivalent circuit model using the PSTrace 5.8 software, where R = resistance and Q = constant phase element. The R 2 circuit element was plotted.

The plasma jet parameters were:

Argon Single Plasma Jet

| | |
|---|---|
| Vpp: | 8 kV |
| Frequency: | 23.5 kHz |
| Gas flow rate: | 1 SLPM |
| Working dist.: | 3~4 mm |
| Treatment time: | 3 mins |

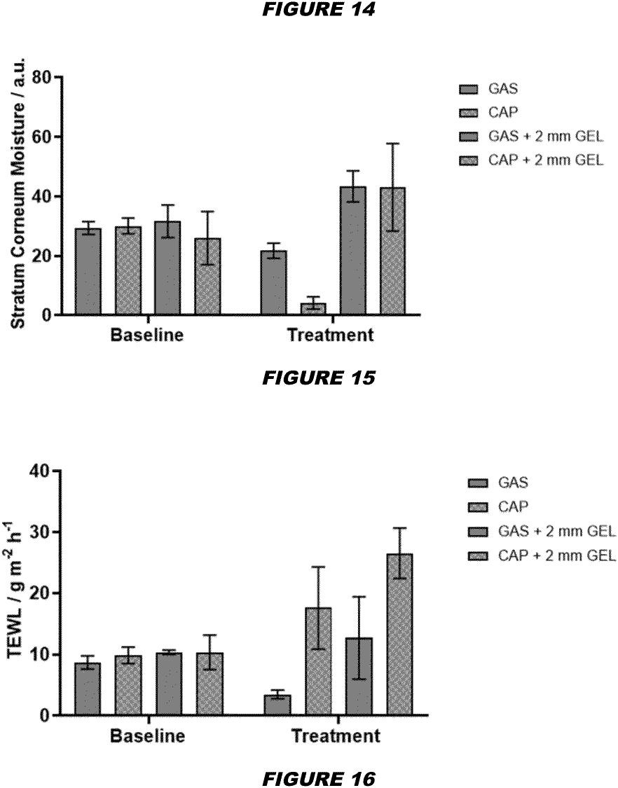

FIG. 14 shows on the left-hand side four separate samples of skin that are all untreated and the sample-to-sample variation in resistance is between different skin samples. On the right-hand side there are four different treatments, GAS only (no plasma) which shows no change in resistance, direct plasma (CAP) (no gel) which shows a log 3-4 change, GAS (no plasma) + hydrogel which shows no change, and CAP + hydrogel which shows no/little change. The decrease in resistance is taken as “damage” to the skin’s architecture.

Moisture and trans epithelial water loss were also measured and the results are shown in FIGS. 15 and 16.

It will be appreciated by those skilled in the art that the invention is not restricted in its use to the particular application described. Neither is the present invention restricted in its preferred embodiment with regard to the particular elements and/or features described or depicted herein. It will be appreciated that the invention is not limited to the embodiment or embodiments disclosed, but is capable of numerous rearrangements, modifications and substitutions without departing from the scope of the invention as set forth and defined by the following claims.

### REFERENCES

Barekzi N, Laroussi M., Plasma Process Polym. 2013 10, 1039-50.

Boateng, Matthews, Stevens and Eccleston, Journal of Pharmaceutical Sciences, 2008 97, 2892-2923.

Boateng, M.K., Journal of Applied Microbiology, 2011, Volume111, Issue 6, Pages 1533-1543.

Djimeli C.L. et al., International Journal of Bacteriology Volume 2014, Article ID 121367, 13 pages

Fridman G, Peddinghaus M, Balasubramanian M, Ayan H, Fridman A, Gutsol A., Plasma Chem Plasma Process., 2006 26, 425-42.

Halliwell B., Gutteridge J.M.C., Free radicals in biology and medicine, 4^{th} Edition Oxford University Press, 2007.

Heinlin J, Isbary G, Stolz W, Morfill G, Landthaler M, Shimizu T, Journal of the European Academy of Dermatology and Venereology, 2011 25, 1-11.

Isbary et al, British Journal of Dermatology, 2010 163, 78-82.

Isbary et al, British Journal of Dermatology, 2012 167, 404-410

Jones and Vaughan, Journal of Orthopaedic Nursing, 2005 9, S1-S11.

Kim, Yelim, et al., Environmental Science & Technology 2022 56 (9), 5763-5774

Kong M G, Kroesen G, Morfill G, Nosenko T, Shimizu T, Dijk J v and Zimmermann J L., New J. Phys., 2009 11, 115012.

Lee HW, Kim GJ, Kim JM, Park JK, Lee JK, Kim GC., J Endod., 2009 35, 587-91.

Nyskohus LS, Watson AJ, Margison GP, Le Leu RK, Kim SW, Lockett TJ., Mutation Research/Genetic Toxicology and Environmental Mutagenesis, 2013 758, 80-6.

Saxon, E. and Peng, X., Chem Bio Chem, 2022, 23, e202100366.

Stoffels E, Sakiyama Y and Graves D B., IEEE Trans. Plasma Sci., 2008 36, 1441.

Sun, Environ. Sci. Technol. 2020, 54, 10, 6427.

Szili EJ, Ghimire B, Patenall BL, Rohaim M, Mistry D, Fellows A, Muhammad M, Jenkins ATA, Short RD. Appl Phys Lett. 2021;119:1-5.

Tonoyan, Front. Microbiol., 2017 Sec. Antimicrobials, Resistance and Chemotherapy

doi.org/10.3389/fmicb.2017.00680.

Vadukoot A.K. et al, Bioorg Med Chem. 2014 December 15; 22(24): 6885-6892.

Throughout the specification and the claims that follow, unless the context requires otherwise, the words “comprise” and “include” and variations such as “comprising” and “including” will be understood to imply the inclusion of a stated integer or group of integers, but not the exclusion of any other integer or group of integers.

The reference to any prior art in this specification is not, and should not be taken as, an acknowledgement of any form of suggestion that such prior art forms part of the common general knowledge.

1. A screen for reducing the concentration of one or more species in plasma, said screen comprising a hydrogel.

2. The screen according to claim 1, wherein the screen reduces the concentration of one or more short lived plasma species from the plasma.

3. The screen according to claim 1, wherein the screen prevents the passage of one or more plasma species or plasma effects from reaching a target site.

4. The screen according to claim 1, wherein the hydrogel is selected from one or more of the group consisting of: gelatin; agarose; hypromellose; Matrigel; extracellular matrix proteins such as fibrin, fibronectin, collagen and collagen derivatives; polysaccharides, such as xanthan gum; sugars; celluloses and modified celluloses such as hydroxypropyl cellulose, sodium carboxymethyl cellulose and hydroxyethyl cellulose; polycarboxylic acids; polyethylene oxide; polyvinyl alcohol; polyacrylic acid; polyvinyl pyrrolidone; polyacrylamidomethylpropanesulfonate; polycaprolactone (PCL); polyglycolic acid (and its derivatives); poly(lactide-co-glycolide); poly(hydroxyalkylmethacrylates); polyurethane-foam; hydrocolloids; and aliginate.

5. The screen according to claim 1 and further comprising a therapeutic agent.

6. The screen according to claim 5, wherein the therapeutic agent is selected from one or more of the group consisting of antibiotics, antiseptic agents, antihistamines, hormones, steroids, therapeutic proteins, molecules, biologics, antibodies, anti-microbial peptides, oligonucleotides, RNAs, enzymes, growth factors, nucleic acids, wound healing agents, anti-inflammatory agents, anti-bacterial agents, antibiotics, and anti-viral agents.

7. A plasma treatment method comprising providing a plasma source and a screen comprising a hydrogel and positioning the screen between the plasma source and a surface of a target to be treated with the plasma such that substantially all of the plasma from the plasma source passes through the screen prior to contacting the surface of the target and the screen reduces the concentration one or more species from the plasma.

8. The plasma treatment method according to claim 7, wherein the plasma is a non-thermal plasma or is operated to produce a plasma having a temperature of less than about 60° C.

9. The plasma treatment method according to claim 7, wherein the screen reduces the concentration of one or more of: UV/VUV radiation, reactive oxygen species (ROS), and reactive nitrogen species (RNS).

10. The plasma treatment method according to claim 7, wherein the screen reduces one or more effects of the plasma on the target.

11. The plasma treatment method according to claim 7, wherein the hydrogel is in the form of a coating on a gauze pad, nonwoven sponge, rope and/or strip.

12. The plasma treatment method according to claim 7, wherein the hydrogel is selected from one or more of the group consisting of: gelatin; agarose; hypromellose; Matrigel; extracellular matrix proteins such as fibrin, fibronectin, collagen and collagen derivatives; polysaccharides, such as xanthan gum; sugars; celluloses and modified celluloses such as hydroxypropyl cellulose, sodium carboxymethyl cellulose and hydroxyethyl cellulose; polycarboxylic acids; polyethylene oxide; polyvinyl alcohol; polyacrylic acid; polyvinyl pyrrolidone; polyacrylamidomethylpropanesulfonate; polycaprolactone (PCL); polyglycolic acid (and its derivatives); poly(lactide-co-glycolide); poly(hydroxyalkylmethacrylates); polyurethane-foam; hydrocolloids; and aliginate.

13. The plasma treatment method according to claim 7, when used for wound treatment.

14. A plasma treatment method comprising providing a plasma source and a screen comprising a hydrogel and a therapeutic agent and positioning the screen between the plasma source and a surface of a target to be treated with the plasma such that substantially all of the plasma from the plasma source passes through the screen prior to contacting the surface of the target and the screen reduces the concentration of one or more species from the plasma and activation of the screen by the plasma results in release of the therapeutic agent onto the surface of the target.

15. The plasma treatment method according to claim 14, wherein the therapeutic agent (a) works in combination with the plasma treatment and/or (b) is released from the hydrogel upon plasma treatment and/or (c) enhances the plasma treatment.

16. The plasma treatment method according to claim 14, comprising multiple activations of the screen over time so as to release the therapeutic agent in stages.

17. The plasma treatment method according to claim 14, wherein the screen is loaded with an agent that on direct or remote plasma activation enhances reactive oxygen species (ROS) production.

18. The plasma treatment method according to claim 14, wherein the therapeutic agent is selected from one or more of the group consisting of antibiotics, antiseptic agents, antihistamines, hormones, steroids, therapeutic proteins, molecules, biologics, antibodies, anti-microbial peptides, oligonucleotides, RNAs, enzymes, growth factors, nucleic acids, wound healing agents, anti-inflammatory agents, anti-bacterial agents, antibiotics, and anti-viral agents.

19. The plasma treatment method according to claim 14, wherein the screen is loaded with a prodrug that is unreactive until oxidized by hydrogen peroxide derived from plasma activation.

20. A therapeutic gel composition comprising a gel forming material and a liquid phase comprising plasma activated liquid.
